(19) United States
(12) Patent Application Publication
(10) Pub. No.: US 2026/0293888 A1
(43) Pub. Date: Oct. 1, 2026

(54) METHODS OF TREATING MOLD

(71) Applicant: Grignard Pure LLC, Rahway, NJ (US)

(72) Inventor: Emile Etienne Grignard III, Long Branch, NJ (US)

(21) Appl. No.: 19/474,861

(22) PCT Filed: Apr. 19, 2024
(86) PCT No.: PCT/US2024/025386

§ 371 Date: Oct. 13, 2025

Related U.S. Application Data
(60) Provisional application No. 63/460,759, filed on Apr. 20, 2023.

(51) Int. Cl. A01N 31/02; A01N 25/06; A01P 3/00; A61L 9/14; A61L 101/34

(52) U.S. Cl. CPC A01N 31/02; A01N 25/06; A01P 3/00; A61L 9/14; A61L 2101/34

(57) ABSTRACT

This disclosure relates to a method for reducing a concentration of mold spores in a space. The method includes treating a space containing mold spores suspended in the air within the space with an effective amount of a sanitizing composition containing triethylene glycol, thereby reducing the concentration of the mold spores suspended in the air within the space.

### CROSS-REFERENCE TO RELATED APPLICATIONS

The present application claims the benefit of U.S. Provisional Application No. 63/460,759, filed Apr. 20, 2023, which is incorporated herein by reference in its entirety.

### TECHNICAL FIELD

This disclosure relates to compositions and methods for treating molds or mold spores in a space (i.e., an outdoor or indoor space).

### BACKGROUND

A mold is one of the structures certain fungi can form. The dust-like, colored appearance of molds is due to the formation of spores containing fungal secondary metabolites. The spores are the dispersal units of the fungi.

Mold spores are a common component of household and workplace dust. When mold spores are present in large quantities, they can present a health hazard to humans, potentially causing allergic reactions and respiratory problems. Some molds also produce mycotoxins that can pose serious health risks to humans and animals. Exposure to high levels of mycotoxins can lead to neurological problems and, in some cases, death. Prolonged exposure (e.g., daily home exposure) can be particularly harmful.

Molds in the home can usually be found in damp, dark or steamy areas, such as bathrooms, kitchens, cluttered storage areas, recently flooded areas, basement areas, plumbing spaces, areas with poor ventilation. Molds can also be found outdoors in humid environments. Symptoms caused by mold allergy are: watery, itchy eyes; a chronic cough; headaches or migraines; difficulty breathing; rashes; tiredness; sinus problems; nasal blockage; and frequent sneezing. Molds can also pose a hazard to human and animal health when they are consumed following the growth of certain mold species in stored food.

Thus, there is a need for improved methods and compositions for treating mold in spaces occupied by human and animals, such as homes, health care facilities, food packaging facilities, and other settings.

### SUMMARY

The inventor surprisingly found that applying (e.g., by atomizing through an atomizing nozzle or vaporizing through a vapor generating device) a composition containing triethylene glycol (TEG) can effectively treat molds or mold spores in the air and/or on the surfaces in a space (i.e., an outdoor or indoor space) in the presence of an active mold source.

In one aspect, this disclosure features a method for reducing a concentration of mold spores in a space, the method including treating a space containing an active mold source with an effective amount of a sanitizing composition containing triethylene glycol, thereby reducing the concentration of the mold spores suspended in the air within the space by at least about 99%.

In another aspect, this disclosure features a method for reducing mold spore transmission, the method including contacting a mold (e.g., on a surface of an article) or a mold spore in a space in the presence of an active mold source with an effective amount of a sanitizing composition containing triethylene glycol, thereby reducing the mold spore transmission by at least about 80%.

In still another aspect, this disclosure features a method for preventing mold spores from forming, the method including treating a space containing an active mold source with an effective amount of a sanitizing composition containing triethylene glycol, thereby preventing generation of mold spores in the space.

Other features, objects, and advantages will be apparent from the description and the claims.

### DETAILED DESCRIPTION

As defined herein, unless otherwise noted, all percentages expressed should be understood to be percentages by weight to the total weight of a composition.

In general, this disclosure relates to compositions and methods for treating molds or mold spores in a space (i.e., an outdoor or indoor space in the presence of an active mold source). As used herein, the term “treat molds or mold spores,” “treating molds or mold spores”, or “treatment of molds or mold spores” can include inactivating molds or mold spores, reducing the amount or concentration of molds or mold spores (e.g., in the air or on a surface), reducing transmission of mold spores, reducing settling of mold spores, and/or preventing a mold from generating mold spores. As used herein, the term “inactivating molds or mold spores” refers to killing molds or mold spores or otherwise make the molds or mold spores inactive. As used herein, the term “active mold source” refers to a live mold capable of generating mold spores.

Examples of suitable spaces that can be treated by the compositions and methods described herein include those in homes, offices, schools, hotels, lobbies, theaters, reception rooms, bathrooms, health care facilities (e.g., nursing homes, hospital rooms (e.g., intensive care facilities), and medical offices (e.g., dental offices)), food packaging facilities, institutional kitchens, cafeterias, restaurants, public transportation vehicles (buses, trains, subways, and airplanes), ambulances, indoor stadiums and athletic facilities, law enforcement facilities (e.g., prisons), government facilities, elevators, retail locations, and other indoor public or private spaces. In general, the sanitizing composition described herein can be used to treat mold or mold spores either in an unoccupied space (e.g., a space not occupied by human or animals) or an occupied space (e.g., a space occupied by human or animals).

In some embodiments, the molds (as well as spores produced from such molds) that can be treated by the sanitizing composition described herein can include Aremonium, Alternaria, Aspergillus, Bolrylis, Cladosporium, Fusarium, Geotrichum, Manoscus, Monila, Mortierella, Mucor, Neurospora, Oidium, Oosproa, Penicilium, Rhizopus, Stachybotrys, Thamnidium, Trichoderma, and Trichophyton.

In some embodiments, this disclosure features a sanitizing composition containing (e.g., comprising, consisting essentially of, or consisting of) triethylene glycol and water (e.g., deionized water). Triethylene glycol is miscible with water, has a boiling point of 286.5° C. at a pressure of 101.325 kPa, and has a relative low vapor pressure compared to water. Without wishing to be bound by theory, it is believed that triethylene glycol treats or inactivates a mold or mold spore by condensing on particles, droplets, or surfaces containing the mold or mold spore until the concentration of triethylene glycol becomes sufficiently high to denature the mold or mold spore. Further, without wishing to be bound by theory, it is believed that triethylene glycol is highly hygroscopic and can inactivate (e.g., kill) a mold or mold spore by absorbing water from the mold or mold spores. In addition, without wishing to be bound by theory, it is believed that triethylene glycol has very low acute or chronic toxicity when inhaled or ingested (especially at the level used in the air to treat (e.g., sanitize or disinfect) an indoor space) and therefore is safe to use in indoor spaces (occupied or unoccupied).

In general, the amount of triethylene glycol in the sanitizing composition described herein is not particular limited and can vary as desired. For example, a sanitizing composition containing a relatively low amount of triethylene glycol can achieve the same disinfection effect against a mold or mold spore as a sanitizing composition containing a relatively high amount of triethylene glycol by applying the former composition in a space at a higher frequency or in a higher amount. In some embodiments, the sanitizing composition described herein can include triethylene glycol in an amount of from at least about 1% (e.g., at least about 5%, at least about 10%, at least about 20%, at least about 30%, at least about 40%, at least about 50%, at least about 52%, at least about 55%, at least about 60%, at least about 65%, at least about 70%, at least about 75%, or at least about 80%) by weight to at most about 99.5% (e.g., at most about 99%, at most about 95%, at most about 90%, at most about 85%, at most about 80%, at most about 75%, at most about 70%, at most about 65%, at most about 60%, or at most about 50%) by weight of the composition. In some embodiments, triethylene glycol can be 100% of the sanitizing composition described herein (i.e., without any other ingredient). It is believed that applying a sanitizing composition containing a relatively high amount (e.g., at least about 50% by weight) of triethylene glycol can increase the efficiency of the disinfection and reduce the frequency of the application of the composition.

In some embodiments, the water in the sanitizing composition described herein is deionized water, reverse osmosis (RO) water, or ultrapure water (e.g., when used in a vaporizer). In some embodiments, the water can have a resistivity of at least 17 mega Ohms, a total organic carbon content of at most about 10 ppb, a bacterial count of at most about 10 CFU/ml). For example, the water can include ions in an amount of from at most about 50 ppm (e.g., at most about 40 ppm, at most about 30 ppm, at most about 20 ppm, at most about 10 ppm, at most about 5 ppm, or at most about 1 ppm) to at least about 1 ppb (e.g., at least about 10 ppb) of the total amount of the water. In some embodiments, when the sanitizing composition described herein is used in connection with an atomizer (e.g., a nebulizer), the water in the composition can be tap water (i.e., not deionized water, RO water, or ultrapure water).

In some embodiments, the sanitizing composition described herein can include water in an amount of from at least about 1% (e.g., at least about 5%, at least about 10%, at least about 15%, at least about 20%, at least about 25%, at least about 30%, at least about 35%, at least about 40%, at least about 45%, at least about 48%, at least about 50%, at least about 60%, or at least about 70%) by weight to at most about 99% (e.g., at most about 95%, at most about 90%, at most about 85%, at most about 80%, at most about 75%, at most about 70%, at most about 65%, at most about 60%, at most about 50%, or at most about 48%) by weight of the composition. Without wishing to be bound by theory, it is believed that using deionized water, RO water, or ultrapure water can minimize clogging the nozzles (e.g., caused by deposition of minerals in water) of the system (e.g., a vaporizer) used to apply the sanitizing composition described herein and therefore can keep the system operating for an extended period of time. In addition, without wishing to be bound by theory, it is believed that the water in the sanitizing composition described herein can facilitate inactivation of molds or mold spores.

Without wishing to be bound by theory, it is believed that including water in the sanitizing composition can allow the composition to be readily nebulized, atomized or vaporized (e.g., by reducing the vaporization temperature and/or increasing the evaporation rate when the sanitizing composition is applied by an atomizer such as a nebulizer, a humidifier, a fog/haze machine, or a smoke generator) and to form an aerosol or vapor in the atmosphere. The water in the aerosol can evaporate rapidly to form fine TEG droplets, which have sanitizing effects and inactivate molds or mold spores in the air or on a surface. In addition, the water in the sanitizing composition can render the composition inflammable, thereby resulting in a safer product than TEG alone (which is a flammable liquid having a flash point of 157° C.).

In some embodiments, the sanitizing composition described herein can further include an optional ingredient, such as a glycol different from triethylene glycol. In some embodiments, the additional glycol can be propylene glycol. Without wishing to be bound by theory, it is believed that the additional glycol can either increase the sanitizing effect of the composition or increase the whiteness of the composition (e.g., to indicate that the sanitizing composition is present in the air). In some embodiments, the sanitizing composition described herein does not include any additional glycol or any components other than triethylene glycol and water.

In some embodiments, the sanitizing composition described herein can include an additional glycol (e.g., propylene glycol (PG)) in an amount of from at least about 0.5% (e.g., at least about 1%, at least about 1.5%, at least about 2%, at least about 2.5%, at least about 3%, at least about 3.5%, at least about 4%, at least about 4.5%) by weight to at most about 99% (e.g., at most about 95%, at most about 90%, at most about 80%, at most about 70%, at most about 60%, at most about 50%, at most about 40%, at most about 30%, at most about 20%, at most about 10%, at most about 5%, at most about 4.5%, at most about 4%, at most about 3.5%, at most about 3%, at most about 2.5%, at most about 2%, at most about 1.5%, or at most about 1%) by weight of the composition.

In some embodiments, sanitizing composition described herein can optionally include a fragrance. Examples of suitable fragrances can include essential oils, esters (e.g., lactones), alcohols, thiols, aldehydes, ketones, amines, hydrocarbons, and aromatic compounds.

In some embodiments, the sanitizing composition described herein can include a fragrance in an amount of from at least about 0.1% (e.g., at least about 0.2%, at least about 0.3%, at least about 0.4%, or at least about 0.5%) by weight to at most about 1% (e.g., at most about 0.9%, at most about 0.8%, at most about 0.7%, at most about 0.6%, or at most about 0.5%) by weight of the composition.

In some embodiments, the sanitizing composition described herein can optionally include a material generally recognized as safe (“GRAS”) as defined by the U.S. Food and Drug Administration. Examples of suitable GRAS materials include dimethyl ether, glycerin, chlorine dioxide, and hypochlorus acid.

In some embodiments, the sanitizing composition described herein can include (e.g., comprise, consist essentially of, or consist of) from about 50% to about 90% by weight triethylene glycol and from about 10% to about 50% by weight water. In some embodiments, the sanitizing composition described herein can include (e.g., comprise, consist essentially of, or consist of) (1) triethylene glycol in an amount of from about 52% to about 90% by weight of the composition; (2) water in an amount of from about 5% to about 48% by weight of the composition; and (3) propylene glycol in an amount of from about 0% to about 5% (e.g., from about 0.5% to about 5%) by weight of the composition. In some embodiments, the sanitizing composition described herein can include (e.g., comprise, consist essentially of, or consist of) about 52.25% by weight triethylene glycol, about 1% by weight propylene glycol, and about 46.75% by weight water.

In some embodiments, this disclosure also features a method of treating molds or mold spores in a space (i.e., an outdoor or indoor space). In some embodiments, the method can be a method for reducing a concentration of mold spores in a space. In some embodiments, such a method can include treating a space containing mold spores suspended in the air within the space by applying (e.g., dispersing, spraying, nebulizing, atomizing, or vaporizing) an effective amount of a sanitizing composition described herein into the space. In some embodiments, applying the sanitizing composition can be performed by a system that generates fog, smoke, or haze, such as a vaporizer (e.g., a smoke generator), a nebulizer (e.g., a scent dispersion unit), or an atomizer (e.g., a humidifier). The system can be those known in the art, such as the fog/haze machines or smoke simulators used in emergency training or used in the lighting industry to generate theatrical effects.

In some embodiments, the method can further include identifying a space in need of treatment of molds or mold spores (e.g., in need of reduction of the amount or concentration of molds or mold spores). For example, a space can be considered in need of treatment of molds or mold spores if it includes a flooded area, a damp area, a steamy area, or an area lacking ventilation. As another example, a space can be considered in need treatment of molds or mold spores if its humidity is at least about 35% (e.g., at least about 40%, at least about 45%, at least about 50%, at least about 55%, at least about 60%, at least about 65%, at least about 70%, at least about 75%, or at least about 80%) and/or at most about 100% (e.g., at most about 95% or at most about 90%).

In some embodiments, the space to be treated can include an active mold source. Without wishing to be bound by theory, it is surprising that the sanitizing composition described herein can effectively treat molds or mold spores (e.g., reducing the concentration of mold spores, reducing mold spore transmission, or preventing mold spores from forming) for an extended period of time even in the presence of an active mold source (which can generate mold spores).

In some embodiments, the method can further include vaporizing the sanitizing composition (e.g., in a humidifier, a fog/haze machine, or a smoke generator) before applying (e.g., spraying) the composition into a space. In some embodiments, vaporizing the composition can be performed by treating the composition with steam or heating. For example, when vaporizing the composition is performed by heating, the method can include delivering the composition to a heat exchanger to vaporize the composition. The heated vapor can be forced through a nozzle as vapor and/or liquid droplets (or liquid particles) to form a visible or invisible aerosol, fog, smoke, or haze. For example, when the sanitizing composition described herein is applied (e.g., dispersed or sprayed) into a space using a vaporizer, the vaporizer can have a liquid reservoir and can use an electric pump to propel the sanitizing composition in the liquid reservoir into a heat exchanger where the sanitizing composition is vaporized. The heated vapor can be forced through a nozzle as vapor and liquid droplets (or liquid particles) that form an opaque fog, smoke, haze, or visible or non-visible aerosol.

In some embodiments, when the sanitizing composition is applied (e.g., dispersed or sprayed) into a space using an atomizer (e.g., a nebulizer), the composition can be converted to an aerosol by pressure. For example, the composition can be pumped into a series of specific sized chambers in an atomizer or nebulizer to increase the pressure and velocity of the composition in order to convert it from a liquid to an aerosol. In some embodiments, ultrasonic and/or vibrating mesh technology can be used to apply the composition into a space through an atomizer or a nebulizer.

In some embodiments, applying the sanitizing composition can form vapor and/or liquid droplets (or liquid particles) that contain triethylene glycol. In some embodiments, the liquid droplets can form an aerosol that contains triethylene glycol. In some embodiments, applying the composition can form an aerosol, a vapor, or a mixture thereof. In some embodiments, the aerosol liquid droplets can have an average diameter of from at least about 10 nm (e.g., at least about nm, at least about 50 nm, at least about 100 nm, at least about 200 nm, at least about 500 nm, at least about 1 μm, at least about 2 μm, or at least about 5 μm) to at most about 10 μm (e.g., at most about 8 μm, at most about 6 μm, at most about 5 μm, at most about 4 μm, at most about 2 μm, at most about 1 μm). In some embodiments, the method described herein can generate from at least about 2000 (e.g., at least about 3000, at least about 4000, at least about 5000, at least about 6000, at least about 8000, or at least about 10,000) to at most about 100,000 (e.g., at most about 50,000 or at most about 25,000) liquid droplets per cm³ of the space (i.e., an outdoor or indoor space).

In some embodiments, applying (e.g., dispersing or spraying) the sanitizing composition can be performed intermittently (e.g., either at a constant interval or at irregular intervals). In some embodiments, when the composition is applied intermittently at a constant interval, the frequency of the application can vary as desired depending on factors such as the concentration of triethylene glycol in the composition, the temperature and humidity of the space, the size of the space, the desired concentration of the composition in the space, and the air exchange rates of the space. In some embodiments, the preferred temperature of the space can range from about 5° C. to about 50° C. (e.g., from about 10° C. to about 30° C. or from about 15° C. to about 30° C.). In some embodiments, the preferred relative humidity of the space can range from about 5% to about 75% (e.g., from about 15% to about 70%, from about 30% to about 65%, or from about 45% to about 60%). In some embodiments, the time period between two applications of the composition can be from at least about 10 seconds (e.g., at least about 30 seconds, at least about 1 minute, at least about 1.5 minutes, at least about 2 minutes, at least about 5 minutes, at least about 10 minutes, at least about 30 minutes, or at least about 1 hour) to at most about 2 hours (e.g., at most about 1 hour, at most about 30 minutes, at most about 10 minutes, or at most about 5 minutes).

In some embodiments, the concentration of the sanitizing composition in an aerosol form (e.g., the concentration of the liquid droplets containing the sanitizing composition) in a space can be from at least about 0.01 mg/m³ (e.g., at least about 0.02 mg/m³, at least about 0.04 mg/m³, at least about 0.05 mg/m³, at least about 0.1 mg/m³, at least about 0.2 mg/m³, at least about 0.3 mg/m³, at least about 0.4 mg/m³, at least about 0.5 mg/m³, at least about 0.6 mg/m³, at least about 0.8 mg/m³, at least about 1 mg/m³, at least about 1.5 mg/m³, at least about 2 mg/m³, at least about 2.5 mg/m³, at least about 3 mg/m³, at least about 3.5 mg/m³, at least about 4 mg/m³, at least about 4.5 mg/m³, or at least about 5 mg/m³) or at most about 10 mg/m³ (e.g., at most about 9 mg/m³, at most about 8 mg/m³, at most about 7 mg/m³, at most about 6 mg/m³, at most about 5 mg/m³, at most about 4 mg/m³, at most about 3 mg/m³, at most about 2 mg/m³, at most about 1 mg/m³, at most about 0.7 mg/m³, at most about 0.5 mg/m³, at most about 0.3 mg/m³, or at most about 0.1 mg/m³). For example, the concertation of the sanitizing composition in an aerosol form (e.g., the concentration of the liquid droplets containing the sanitizing composition) in a space can be from about 0.02 mg/m³ to about 1.6 mg/m³ (e.g., from about 0.04 mg/m³ to about 1 mg/m³).

In some embodiments, the total concentration of the TEG (including TEG in the aerosol and TEG in the vapor) or glycol (e.g., all glycols in the aerosol and in the vapor) in a space can be from at least about 0.01 mg/m³ (e.g., at least about 0.02 mg/m³, at least about 0.04 mg/m³, at least about 0.05 mg/m³, at least about 0.1 mg/m³, at least about 0.2 mg/m³, at least about 0.3 mg/m³, at least about 0.4 mg/m³, at least about 0.5 mg/m³, at least about 0.6 mg/m³, at least about 0.8 mg/m³, at least about 1 mg/m³, at least about 1.5 mg/m³, at least about 2 mg/m³, at least about 2.5 mg/m³, at least about 3 mg/m³, at least about 3.5 mg/m³, at least about 4 mg/m³, at least about 4.5 mg/m³, or at least about 5 mg/m³) or at most about 10 mg/m³ (e.g., at most about 9 mg/m³, at most about 8 mg/m³, at most about 7 mg/m³, at most about 6 mg/m³, at most about 5 mg/m³, at most about 4 mg/m³, at most about 3 mg/m³, at most about 2 mg/m³, at most about 1 mg/m³, at most about 0.8 mg/m³, at most about 0.6 mg/m³, at most about 0.5 mg/m³, at most about 0.3 mg/m³, or at most about 0.1 mg/m³). For example, the total concertation of the TEG (including TEG in the aerosol and TEG in the vapor) or glycol (including all glycols in the aerosol and in the vapor) in a space can be from about 0.01 mg/m³ to about 3 mg/m³ (e.g., e.g., from about 0.5 mg/m³ to about 3 mg/m³ or from about 0.5 mg/m³ to about 1 mg/m³).

In some embodiments, the method includes maintaining the concentration of the sanitizing composition in an aerosol form (e.g., in the form of aerosol particles) or the concentration of TEG within the above ranges for a period of time from at least about 30 minutes (e.g., at least about 1 hour, at least about 2 hours, at least about 4 hours, at least about 5 hours, at least about 6 hours, at least about 9 hours, or at least about 12 hours) to at most about 24 hours (e.g., at most about 21 hours, at most about 18 hours, at most about 15 hours, at most about 12 hours, at most about 9 hours, or at most about 6 hours) per day. In some embodiments, the method includes treating an indoor space with the sanitizing composition described herein for at least two days (e.g., at least three days, at least four days, at least five days, at least six days, at least eight days, at least ten days, at least 15 days, at least 20 days, at least 30 days, at least one month, at least two months, at least three months, or at least six months) or continuously until the mold is believed to be effectively treated or treatment is no longer needed. In some embodiments, the treatment days can be consecutive, in a certain frequency (e.g., every other day), or random within a period of time (e.g., a week, a month, three months, or a year). Without wishing to be bound by theory, it is believed that maintaining the sanitizing composition or the TEG at a concentration within the above ranges for a relatively short period of time (e.g., about 6 hours) can reduce the concentration of mold spores in a space containing a viable mold by at least about 96% (e.g., at least about 97%, at least about 98%, at least about 99%, at least about 99.5, or at least about 99.9%). As used herein, the concentration reduction of mold spores refers to their gross concentration reduction, which includes both the concentration reduction by natural die-off of the mold spores and the concentration reduction of the mold spores caused by the sanitizing composition described herein.

Without wishing to be bound by theory, it is believed that the concentration reduction of mold spores in the air by natural settling in an indoor space containing a viable mold may not reach 96% net reduction if the space is not sanitized as the mold can continue to generate mold spores. However, without wishing to be bound by theory, it is believed that, in some embodiments, if the sanitizing composition described herein is utilized on a daily basis not less than three days, 2 hours per day, the airborne mold spore concentration will be reduced by at least 96% for a relatively long period of time. In some embodiments, at least about 0.1 gram (e.g., at least about 0.5 gram, at least about 1 gram, at least about 2 grams, at least about 3 grams, or at least about 4 grams) and/or at most about 5 grams of the sanitizing composition can be used in a space having a volume of 1000 cubic feet every 6 hours (e.g., every 4 hours, every 2 hours, or every 1 hour).

In some embodiments, this disclosure features a method for reducing mold spore transmission. In some embodiments, the method can include contacting a mold (e.g., a mold on a surface of an article in the space) or a mold spore (e.g., a mold spore in the air of the space) with an effective amount of a sanitizing composition containing triethylene glycol described herein, thereby reducing the mold spore transmission. As used herein, the term “mold spore transmission” includes both travelling of mold spore in the air and settling of mold spore on a surface. In some embodiments, the method can reduce mold transmission by at least about 80% (e.g., at least about 85%, at least about 90%, at least about 95%, at least about 98%, at least about 99%, at least about 99.5, or at least about 99.9%) or about 100%. In some embodiments, the method for reducing mold spore transmission can be performed under conditions (e.g., treatment time, aerosol concentration, aerosol particle size, and TEG concentration) or in a manner similar to those described above with respect to the method for reducing a concentration of mold spores. Without wishing to be bound by theory, it is believed that the above method can reduce mold spore transmission by inactivating the mold within the space (which would prevent the mold from producing mold spores) or by inactivating the mold spores in the air of the space directly.

In some embodiments, the method can reduce settling of mold spores on a surface by at least about 80% (e.g., at least about 85%, at least about 90%, at least about 95%, at least about 98%, at least about 99%, at least about 99.5, or at least about 99.9%) or about 100%. In general, the surface can be any surface of an article in the space exposed to the sanitizing composition described herein. The surface can be a porous surface or a non-porous surface (e.g., a hard surface).

In some embodiments, this disclosure features a method for preventing mold spore from forming. In some embodiments, the method can include treating a space with an effective amount of a sanitizing composition containing triethylene glycol described herein, thereby preventing generation of mold spores in the space. In some embodiments, the space includes a mold. In such embodiments, the method can prevent the existing mold from producing mold spores. In some embodiments, the space does not include a mold. In such embodiments, the method can prevent a viable mold or mold spore from being transmitted into the space. In some embodiments, the method for preventing mold spore from forming can be performed under conditions (e.g., treatment time, aerosol concentration, aerosol particle size, and TEG concentration) or in a manner similar to those described above with respect to the method for reducing a concentration of mold spores. Without wishing to be bound by theory, it is believed that the above method can prevent mold spore from forming by inactivating the mold within the space (which would prevent the mold from producing mold spores).

In some embodiments, to practice the sanitizing methods described herein in an indoor space, one can place a system described herein (e.g., a vaporizer or an atomizer such as a nebulizer) in the center or on one or more sides of the indoor space to be treated. In some embodiments, multiple systems can be used at appropriate places to ensure even distribution of the sanitizing composition. The sanitizing composition described herein can be applied from the system(s) into the indoor space until a desired sanitization (or disinfection) level is achieved. In some embodiments, the sanitizing composition can be applied intermittently (e.g., every 2 minutes or every 30 minutes) to maintain the desired sanitization level for a pre-determined period of time (e.g., 6 hours per day).

In some embodiments, the sanitizing composition described herein can be applied to an indoor space to be treated via an HVAC unit. For example, a system containing the composition described herein can be connected to the return plenum of an HVAC unit through a tubing. The composition can then be applied into the indoor space through the HVAC unit until a desired sanitization (or disinfection) level is achieved. This approach can sanitize both the filter in the HVAC unit and the indoor space.

In some embodiments, the space (e.g., the indoor space) to be treated can include a mold spore suspending in the air and the sanitizing method described herein is capable of inactivating (e.g., killing) the mold spore in the air. In some embodiments, the space can include a mold or mold spore on a surface (e.g., either a hard or soft surface, or either a non-porous or a porous surface) and the sanitizing method described herein is capable of inactivating (e.g., killing) the mold or mold spore on the surface. In some embodiments, the surface can be any surface in an indoor space, such as a surface of a wall, a floor, a desk, a chair, a computer, a rug, or a drape. Without wishing to be bound by theory, it is believed that triethylene glycol can adhere to the mold or mold spore either in the air or on a surface to inactivate the mold or mold spore by desiccation

In another aspect, this disclosure features a packaged product that includes a container (e.g., a can or a bottle), and the sanitizing composition described herein in the container. The packaged product can be either pressurized or non-pressurized.

The following examples are illustrative and not intended to be limiting.

### EXAMPLES

### Example 1: Efficacy Study Using Time Controlled Release of Sanitizing Composition #1 by Against Aspergillus Brasiliensis in the Presence of Active Source

Sanitizing composition #1 was tested against the spores of Aspergillus brasiliensis (a mold) in an aerosol in the presence of an active Aspergillus brasiliensis source by using an Aura diffuser based on the following procedure.

A summary of testing conditions is provided below:
Test Substance: Sanitizing Composition #1: triethylene glycol (52.25 wt %), propylene glycol (1 wt %), and DI water (46.75%)
Test Substance Aerosol Concentration: 0.1 mg/m³.
Daily treatment duration: 6 hours
Treatment period: 6 days
Test Organism: Aspergillus brasiliensis
Starting powder stock concentration: 1.0E+09 cfu/g
Mold dispersal time: 2 Minutes
Chamber Size: 1 m³
Exposure Temperature: 22-24° C.
Exposure Humidity: 60-70% Relative Humidity
Aerosol Sampling Duration: 10 minutes
Aerosol Sampling Time Points: 0-hour, 24-hour, 30-hour, 48-hour, 54-hour, 72-hour, 78-hour, 96-hour, 102-hour, and 120-hour

### Recovery Media

Neutralizer: Phosphate buffered saline solution+0.05% Tween 80
Agar Plate Medium: Potato Dextrose Agar

The testing of the treatment of an active mold source by Sanitizing Composition #1 over a 6-day period consisted of two tests happening simultaneously in the testing chamber. These tests were (1) bioaerosol reduction testing and (2) mold deposition testing. The bioaerosol reduction testing was conducted by aerosolizing mold off of an active mold source and sampling the chamber air to characterize the mold concentration in the chamber. This sampling occurred before and after each daily treatment. The mold deposition testing was conducted by exposing glass slides to the active mold for 24 hours at a time to evaluate the effect of the treatment of Sanitizing Compound #1 on the amount of mold deposited. Six glass slides were placed in the test chamber and one slide was removed each day during the 6-day testing period. These tests were performed twice under the same conditions as Trial 1 and Trial 2.

### Chamber Preparation

Prior to the initiation of testing, and between each trial, the chamber was wiped clean with 95% Isopropyl alcohol using a non-fibrous towel. All ports were cleaned with non-fibrous brushes. All surfaces were allowed to dry before the trial commencement. The lot number of the cleaning solution, as well as the time and date of the cleaning, were recorded for each cleaning of the chamber.

### Preparation of Test Organism

A. brasiliensis mold spores were obtained in purified bulk powder form at a concentration of 1×10⁹ cfu/g. To verify the bulk powder spore concentration, an aliquot of weighed dry powder was prepared in suspension in PBS+0.005% Tween 80 at a mass:volume ratio to obtain a concentration of 1×10⁹ cfu/ml. This aliquoted spore suspension was plated, prior to testing, to verify the concentration. It was also streaked to check for purity of the stock. The purified bulk powder was retrieved from the freezer prior to each trial. For each trial, the spore stock was diluted to 1×10⁴ cfu/mL in PBS. Using this diluted spore stock six (6) tryptic soy agar plates were coated with 750 μl of spore solution. The 750 μl was spread evenly across the entire plate before being allowed to dry. After drying, the 6 spore plates were placed into a sealable bag and moved into the incubator at 30° C. The mold plates were allowed to grow for 5 days before use in the test trial. After 5 days, the bag of plates was moved from the incubator into the 1 m³ chamber.

### Neutralization Verification

Neutralization verification testing was run to assure that all kill assessed to the test device was due to kill in the air and not kill in the neutralizer PBS solution after sampling and during plating.

For neutralization verification testing, the amount of Sanitizing Composition #1 was calculated based on a 10-minute sample at the desired aerosol concentration with an impinger pulling 12.5 l/min. To verify that any fluctuations of that concentration wouldn't cause an issue, the verification testing was also done at 1 log (10%) and 2 log (100%) higher concentrations of Sanitizing Composition #1 than were estimated to be in the chamber. These concentrations of Sanitizing Composition #1 were diluted in PBS+0.005% Tween 80. The A. brasiliensis powder stock used for testing was diluted to a concentration below 1.00×10³ pfu/mL and added to each conical tube. The contact time in the PBS mixtures for testing was 15 minutes. After 15 minutes, all samples were diluted and plated using the sample plating method described below, incubated, and enumerated for concentration. The concentration of each sample was compared to that of the control with only PBS solution and a control with the sample diluted in sterile deionized water to confirm neutralization.

### Chamber Testing

Aerosolization testing for Sanitizing Composition #1, against an active A. brasiliensis source, consisted of a single control and duplicate test trials. The control was run to account for any natural die off of the organism over time in the chamber.

Along with the aerosol testing, blank glass slides were placed in the chamber to assess the amount of settling yielded by the microorganism. 1″×2″ glass slides were placed on the opposite side of the chamber from the mold plates and 2 ft. from the Aura diffuser. To prepare the Aura diffuser for testing with the Sanitizing Composition #1 solution, the reservoir of the Aura diffuser was filled with the Sanitizing Composition #1 solution. The Aura diffuser was adjusted to settings determined in the concentration matching section of the protocol. Before being placed into the chamber, the Aura diffuser was primed. Priming the Aura diffuser consisted of running it outside of the chamber for 20 minutes. This 20-minute prime ensured that the lines of the Aura diffuser were full of fresh Sanitizing Composition #1 solution and that the Aura diffuser was running correctly.

Prior to the start of testing, the Aura diffuser was placed on the floor in the center of the chamber. The Aura diffuser was checked to ensure the proper settings were still enabled before the door of the chamber was closed. After the chamber door was sealed, all outlets of the chamber were checked, all vacuum pumps were verified to be working, and the pressurized air was verified as functioning. After the checks, using the glove ports, the mold plates were opened and exposed to the chamber air. The mold plates were lined up in a 2×3 formation in front of a dispersal fan. Each day an initial concentration sample was taken by turning on the dispersal fan and the chamber mixing fans for 2 minutes. Following the 2 minutes, the chamber air was sampled for 10 minutes. After the completion of the aerosol sample, the Aura diffuser was turned on. The Aura diffuser was set to maintain the concentration of Sanitizing Composition #1 in the chamber at 0.1 mg/m³ throughout each treatment. Each day the Aura diffuser was set to treat the chamber for 6 hours on a timed schedule. After the treatment, the sampling procedure was repeated to produce a post treatment concentration.

### Aerosol Collection

Samples were collected using AGI-30 impingers or viable cascade bio impactors. The AGI-30 impingers were filled with 20 mL of sterile PBS with the addition of 0.005% Tween 80. Tween 80 was used to increase the impinger collection efficiency and for the de-agglomeration of all microorganisms. Sampling was done from opposite corners of the chamber simultaneously. After sampling, the impingers were pooled and collected in conical tubes for plating. The impinger sampling rate was 12.5 l/min using suction from the vacuum pumps. Each impinger used in the study was flow characterized prior to the initiation of the study. When using the viable cascade bio impactor, an agar plate was loaded into the impactor prior to beginning sampling. After sampling, the plates were covered and moved directly into the incubator. Aerosol samples were collected before and after treatment with Sanitizing Composition #1 each day.

### Deposition Slide Collection

The deposition slides were removed from the chamber after the completion of the trial and evacuation of the chamber. Each plate was placed in separate individually labeled 50 mL conical tubes containing 10 mL of sterile phosphate buffered saline solution+tween 80 to be plated to determine the concentration. Prior to plating, each conical tube was vortexed for 2 minutes and then vigorously shaken for another minute. The samples were then plated using the technique described in the Sample Plating portion below for concentration determination.

### Sampler Cleaning

Between each sample, the impingers or the cascade impactors were submerged in a 10% bleach solution and then in DI water. They were then washed with high pressure tap water in a sink for a total of six times followed by a final DI water rinse. They were then air dried before being used for another sample.

### Sample Plating

For sample plating, 1.5 mL micro centrifuge tubes were filled with 900 μL of sterile PBS for dilution of the samples. The number of tubes needed was based on the number of samples to be plated and the predicted dilution ranges needed to plate the samples. Using the set of dilution tubes, serial dilutions were performed with the test samples. For this study, a standard small drop plaque assay technique was instituted for plating. The countable range for this assay was 5-50 plaques. Counts >50 cfu were labeled as too numerous to count (TNTC). The samples were plated in their respective dilution ranges in triplicate on pre-labeled culture plates. The plates were allowed to dry, then placed in the incubator set at 20-25° C. for 48 hours.

### Incubation and Observation

The plates were allowed to incubated at 20-25° C. for 48 hours before being removed for enumeration. Plate counts were recorded in lab notebooks at the time of enumeration and transferred to Microsoft Excel spreadsheets for analysis.

### Study Controls

Control Count: The A. brasiliensis nebulization stock yielded acceptable starting concentration counts of >1.0×10⁷ cfu/ml.

Sterility Control: The media sterility check revealed no growth of a contaminant.

Culture Purity: The host culture showed no signs of contamination when being streak plated on a culture plate.

Neutralization Control: Neutralizer yielded no signs of contaminant growth after being plated.

### Data Analysis

### Calculation of % Reduction and Log10 Reduction

The starting bioaerosol concentrations of the trials were characterized in the first T-0 sample before any treatment with Sanitizing Composition #1 had taken place.

To determine the log reduction of the trial at each time point, the percent of viable mold airborne in the chamber after the aerosolization period was compared to that initial T-0 bioaerosol concentration.

For net reduction, the natural losses from the control trial were subtracted from the test trial to account for natural losses not caused by Sanitizing Composition #1.

The daily settling plates from the control trial were directly compared to the settling plates from Sanitizing Composition #1 trial to assess reduction. The difference remaining between the control trial and Sanitizing Composition #1 test trial was multiplied by the log 10 function to yield the log reduction. Since the natural die off should be the same between the trials, the reduction was assessed as net reduction.

To evaluate the viable aerosol delivery efficiency and define operation parameters of the system, calculations based on (theoretical) 100% efficacy of aerosol dissemination were derived using the following steps and parameters:
Plating and enumeration of the biological to derive the concentration of the stock suspension (Cₛ) cfu/g for dry powder.
Collison 24 jet nebulizer use rate (R_{neb}) (volume of dry powder spores dispersed) at 28 psi air supply pressure.
Aerosol Generation time (t)
Chamber volume (V_{c})

Assuming 100% efficiency, the quantity of aerosolized viable particles (V_{P}) per liter of air in the chamber for a given biological stock concentration (Cₛ) is calculated as:

$${V}_{P} = \frac{{C}_{s} \cdot {R}_{neb}}{{V}_{c}} t$$

### AGI-30 Impinger Collection Calculation:

Viable aerosol concentration collection (Cₐ)=cfu/L of chamber air.
Viable Impinger concentration collection (Cᵢₘₚ)=cfu/mL from enumeration of impinger sample.
Impinger sample collection volume (Iᵥₒₗ)=20 mL collection fluid/impinger.
AGI-30 impinger sample flow rate (Qᵢₘₚ)=12.5 L/min.
AGI-30 impinger sample time (t)=5 or 10 minutes, test dependent.
V_{P} is viable particles in cfu per liter of air as calculated above.

For viable impinger aerosol concentration collection (Cₐ)=cfu/L of chamber air:

$${C}_{a} = \frac{{C}_{Imp} \cdot {I}_{vol}}{{Q}_{imp}} t$$

The aerosol system viable delivery efficiency (expressed as %) is:

$$Efficiency = \frac{{C}_{a}}{{V}_{p}} \cdot 100$$

All runs were averaged to obtain a mean and standard deviation for each trial.

Total viable biological concentrations along with reductions over time (i.e., log reductions of aerosolized biologicals) were calculated and graphed for each trial.

### Test Results

The results of bioaerosol reduction testing in the two trials mentioned above are summarized in Tables 1 and 2 below.

TABLE 1

| | | | Trial 1 | | Control | |
|---|---|---|---|---|---|---|
| Time | Control Conc | Trial 1 | gross % | LOG₁₀ | LOG₁₀ | Net LOG₁₀ |
| (hour) | (cfu/L) | Conc (cfu/L) | Reduction | Reduction | Reduction | Reduction |
| 0 | 5.44E+01 | 5.17E+01 | 0.0% | N/A | N/A | N/A |
| 24 | 5.33E+00 | 4.16E+00 | 92.0% | 1.09 | 1.01 | 0.09 |
| 30 | 3.73E+00 | 1.38E+00 | 97.3% | 1.57 | 1.16 | 0.41 |
| 48 | 2.67E+00 | 2.67E−01 | 99.5% | 2.29 | 1.31 | 0.98 |
| 54 | 2.45E+00 | 1.97E−01 | 99.6% | 2.42 | 1.35 | 1.07 |
| 72 | 5.44E+00 | 5.94E−01 | 98.9% | 1.94 | 1.00 | 0.94 |
| 78 | 1.40E+01 | 6.43E−01 | 98.8% | 1.91 | 0.59 | 1.32 |
| 96 | 1.57E+01 | 1.33E+00 | 97.4% | 1.59 | 0.54 | 1.05 |
| 102 | 1.55E+01 | 3.33E−02 | 99.9% | 3.19 | 0.55 | 2.64 |
| 120 | 2.86E+01 | 3.70E−03 | 100.0% | 4.15 | 0.28 | 3.87 |

TABLE 2

| | | | Trial 2 | | Control | |
|---|---|---|---|---|---|---|
| Time | Control Conc | Trial 2 | gross % | LOG₁₀ | LOG₁₀ | Net LOG₁₀ |
| (hour) | (cfu/L) | Conc (cfu/L) | Reduction | Reduction | Reduction | Reduction |
| 0 | 5.44E+01 | 4.43E+01 | 0.0% | N/A | N/A | N/A |
| 24 | 5.33E+00 | 2.57E−01 | 99.4% | 2.24 | 1.01 | 1.23 |
| 30 | 3.73E+00 | 1.00E−01 | 99.8% | 2.65 | 1.16 | 1.48 |
| 48 | 2.67E+00 | 2.83E−02 | 99.9% | 3.19 | 1.31 | 1.88 |
| 54 | 2.45E+00 | 5.67E−02 | 99.9% | 2.89 | 1.35 | 1.55 |
| 72 | 5.44E+00 | 4.83E−02 | 99.9% | 2.96 | 1.00 | 1.96 |
| 78 | 1.40E+01 | 4.67E−02 | 99.9% | 2.98 | 0.59 | 2.39 |
| 96 | 1.57E+01 | 1.67E−02 | 100.0% | 3.42 | 0.54 | 2.88 |
| 102 | 1.55E+01 | 1.39E−02 | 100.0% | 3.50 | 0.55 | 2.96 |
| 120 | 2.86E+01 | 2.78E−03 | 100.0% | 4.20 | 0.28 | 3.92 |

As shown in Tables 1 and 2, due to the presence of an active mold source, although the airborne mold spore concentration decreased initially (e.g., as a result of natural loss such as settling), it increased after 54 hours. However, despite the increase in the airborne mold spore concentration, Sanitizing Composition #1 unexpectedly exhibited a net Log₁₀ reduction of at least 2.64 after 102 hours against the spores of Aspergillus brasiliensis in the aerosol. In other words, the composition was able to kill more than 99.7% of the spores of Aspergillus brasiliensis in the aerosol after 102 hours. The above results suggest that Sanitizing Composition #1 would be effective in killing mold spores in the aerosol form in a space in the presence of an active mold source.

In addition, the mold deposition test showed that the daily log 10 reductions of Aspergillus brasiliensis settling on a glass slide ranged from 1.30 to 1.93 in Trial 1 and ranged from 1.00 to 1.93 in Trial 2 compared to a control experiment (where Sanitizing Composition #1 was not introduced into the test chamber). In other words, the results showed that Sanitizing Composition #1 was able to reduce the settling of the spores of Aspergillus brasiliensis onto a glass slide by at least 90% each day. The above results suggest that Sanitizing Composition #1 would be effective in preventing and reducing mold spore transmission and in preventing generation of mold spores from an active mold source.

Other embodiments are in the following claims.

1. A method for reducing a concentration of mold spores in a space, comprising:
treating a space containing an active mold source with an effective amount of a sanitizing composition comprising triethylene glycol, thereby reducing the concentration of the mold spores suspended in the air within the space by at least about 96%.

2. The method of claim 1, wherein the mold spores are produced from Aremonium, Alternaria, Aspergillus, Botrytis, Candida auris, Cladosporium, Fusarium, Geotrichum, Monascus, Monilia, Mortierella, Mucor, Neurospora, Oidium, Oosproa, Penicilium, Rhizopus, Stachybotrys, Thamnidium, Trichoderma, and Trichophyton.

3. The method of claim 1, further comprising identifying a space in need of a reduction of the concentration of mold spores.

4. The method of claim 1, wherein the method reduces the concentration of the mold spores suspending in the air within the space by at least about 99.9% after treatment with the sanitizing composition.

5. The method of claim 1, wherein treating the space comprises dispersing the sanitizing composition into the space to form aerosol particles and vapor.

6. The method of claim 5, wherein treating the space comprises maintaining the aerosol particles at a concentration ranging from about 0.02 mg/m³ to about 3 mg/m³ in the space.

7. The method of claim 6, wherein treating the space comprises maintaining the aerosol particles at the concentration from about 30 minutes to about 24 hours per day.

8. The method of any one of claim 5, wherein the aerosol particles have an average diameter of from about 10 nm to about 10 m.

9. The method of claim 1, wherein the triethylene glycol is in an amount of from about 1% to about 90% by weight of the sanitizing composition.

10. The method of claim 1, wherein the sanitizing composition further comprises water.

11. The method of claim 10, wherein the water is in an amount of from about 5% to about 90% by weight of the sanitizing composition.

12. The method of claim 1, wherein the sanitizing composition further comprises a second glycol different from triethylene glycol.

13. The method of claim 12, wherein the second glycol is in an amount of from about 0.5% to about 90% by weight of the sanitizing composition.

14. The method of claim 1, wherein the sanitizing composition comprises from about 1% to about 90% by weight triethylene glycol, and from about 1% to about 90% by weight water.

15. The method of claim 1, wherein the sanitizing composition comprises about 52.25% by weight triethylene glycol, about 1% by weight propylene glycol, and about 46.75% by weight water.

16. The method of claim 1, wherein the sanitizing composition further comprises a fragrance.

17. The method of claim 16, wherein the fragrance is in an amount of from about 0.1% to about 1% by weight of the sanitizing composition.

18. A method for reducing mold spore transmission, comprising:
contacting a mold or a mold spore in a space in the presence of an active mold source with an effective amount of a sanitizing composition comprising triethylene glycol, thereby reducing the mold spore transmission by at least about 80%.

19. The method of claim 18, wherein the method reduces settling of mold spores on a surface by at least about 80%.

20. A method for preventing mold spores from forming, comprising:
treating a space containing an active mold source with an effective amount of a sanitizing composition comprising triethylene glycol, thereby preventing generation of mold spores in the space.

21. The method of claim 20, wherein the space comprises a mold.
